(19) United States
(12) Patent Application Publication
(10) Pub. No.: US 2011/0177180 A1
(43) Pub. Date: Jul. 21, 2011

(54) HERBAL COMPOSITION AND METHOD FOR THE TREATMENT OF VIRAL INFECTION

(75) Inventors: Becky Mary Thomas, Mumbai (IN); Saravanabalaji Shanmugam, Mumbai (IN); Arvind Saklani, Mumbai (IN); Shreekant Nagesh Godbole, Mumbai (IN); Vinayak Raghoba Naik, Mumbai (IN); Prabhu Dutt Mishra, Mumbai (IN); Sivaramakrishnan Hariharan, Mumbai (IN); Arno Appavoo Enose, Mumbai (IN); Ritu Kaushik, Mumbai (IN)

(73) Assignee: PIRAMAL LIFE SCIENCES LIMITED, Mumbai (IN)

(21) Appl. No.: 13/120,765

(22) PCT Filed: Sep. 23, 2009
(86) PCT No.: PCT/IB2009/054168

§ 371 Date: Mar. 24, 2011

Related U.S. Application Data
(60) Provisional application No. 61/100,099, filed on Sep. 25, 2008.

(51) Int. Cl. A61K 36/48; A61P 31/12

(52) U.S. Cl. 424/757

(57) ABSTRACT

The present invention relates to a herbal composition comprising extract of the plant Indigofera heterantha. The invention also relates to a process for the preparation of the herbal composition. The invention further relates to the use of the herbal composition for the treatment of viral infection, particularly caused by herpes simplex viruses.

### FIELD OF THE INVENTION

The present invention relates to a herbal composition comprising an extract of the plant Indigofera heterantha having antiviral activity. The invention also relates to a process for the preparation of the herbal composition. The invention further relates to the use of the herbal composition for the treatment of viral infections, particularly those caused by herpes simplex viruses.

### BACKGROUND OF THE INVENTION

Viruses are the etiological cause of many life threatening or life impairing human diseases. Of special concern are herpes viruses such as herpes simplex virus type 1 (HSV-1), herpes simplex virus type 2 (HSV-2), cytomegalovirus (CMV), Epstein-Barr virus (EBV), varicella zoster virus (VZV), and human herpes viruses 6, 7 and 8 (HHV-6, HHV-7 and HHV-8) and the like.

Herpes simplex is a viral disease caused by herpes simplex viruses (HSV). HSV-1 is commonly associated with facial herpes known as cold sores or fever blisters, whereas HSV-2 is more often associated with genital herpes. Diseases caused by HSV may become life threatening in immunocompromised patients, especially HIV infected patients. After primary infection, HSV persists in the host for the latter's entire lifetime, thus HSV infection is considered as a lifelong infection (The Journal of Infectious Diseases, 2002, 186, S71-S77).

Amongst the antiviral drugs, acyclovir has by far gained the widest acceptance for clinical use. Acyclovir is a guanine analog, which interferes with the DNA polymerase of the virus and thereby inhibits viral DNA replication (Clinical Microbiology Review, 1994, 7 (1), 1-13).

Acyclovir is used for the treatment HSV-1 and HSV-2. The success of acyclovir provided an encouragement in the early 1980s to discover anti-HIV agents and the first one to be licensed for clinical use was azidothymidine (AZT). In the mid 1990s, specific designing of protease inhibitors facilitated a new approach of targeting viral enzymes that was crucial in viral replication (Drug Discovery, 2007, 6, 941).

Methanol extract of whole plant of Indigofera tinctoria is reported to be active against human immunodeficiency virus type 1 (strain HTLV-III_{B}LAI) and human immunodeficiency virus type 2 (strain LAV-2ROD) replicating in acutely infected MT-4 cells (Hamdard Medicus, 2000, vol. 43 (1), 5-7). Alcohol extract of stem of Indigofera aspalathoides is reported to be active against HEL cell culture (herpes simplex virus-1 KOS; herpes simplex virus-2 G; vaccinia virus; vesicular stomatitis virus and herpes simplex virus-TK KOS ACV) and HeLa cell culture (vesicular stomatitis virus, coxsackie virus B4 and respiratory syncytial virus) (Pharmacognosy Magazine, 2007, vol 3, 163-166).

There continues to be a need for effective compositions and methods for the prevention and treatment of viral infections, particularly herpes infections. The incidence and severity of herpes infections have increased due to increase in the number of immunocompromised patients produced by aggressive chemotherapy regimens, expanded organ transplantation and the rising incidence of HIV infections.

### SUMMARY OF THE INVENTION

The present invention relates to a herbal composition comprising a therapeutically effective amount of an extract of the plant Indigofera heterantha either alone or in combination with a pharmaceutically acceptable carrier.

The invention also relates to a process for the preparation of the herbal composition and the extract.

The invention also relates to the antiviral activity of the herbal composition.

An antiviral activity of the composition is anti-HSV activity, particularly anti-HSV-2 activity.

The invention further relates to a method for treating a viral infection in a mammal comprising administering to the mammal a therapeutically effective amount of the herbal composition.

The invention also relates to the use of the herbal composition for the prevention of viral infection with the use of condoms or other barrier devices.

The invention includes the use of the herbal composition for the treatment of viral infection.

The invention also includes the use of the extract of the plant Indigofera heterantha for the manufacture of a medicament for the treatment of viral infection.

### BRIEF DESCRIPTION OF THE DRAWINGS

FIG. 1: Effect of extract of Example 2 on HSV-2 replication and cell viability in Vero cell line.

FIG. 2: Effect of extract of Example 5 on HSV-2 replication and cell viability in Vero cell line.

FIG. 3: Effect of extract of Example 6 on HSV-2 replication and cell viability in Vero cell line.

FIG. 4: Effect of extract of Example 7 (d) on HSV-2 replication and cell viability in Vero cell line.

FIG. 5A: Survival plot of effect of treatment using extract of Example 1 in mouse vaginal model of HSV-2 infection.

FIG. 5B: Extravaginal lesion score of treatment using extract of Example 1 in mouse vaginal model of HSV-2 infection.

FIG. 6A: Survival plot of effect of treatment using Formulation I in mouse vaginal model of HSV-2 infection.

FIG. 6B: Extravaginal lesion score of treatment using Formulation I in mouse vaginal model of HSV-2 infection.

FIG. 7A: Survival plot of effect of treatment using Formulation II in mouse vaginal model of HSV-2 infection.

FIG. 7B: Extravaginal lesion score of treatment using Formulation II in mouse vaginal model of HSV-2 infection.

FIG. 8A: Survival plot of effect of treatment using Formulation III in mouse vaginal model of HSV-2 infection.

FIG. 8B: Extravaginal lesion score of treatment using Formulation III in mouse vaginal model of HSV-2 infection.

### DETAILED DESCRIPTION OF THE INVENTION

Before describing the present invention in detail, it has to be understood that this invention is not limited to particular embodiments. It is also to be understood that the terminology used herein is for the purpose of describing particular embodiments only, and is not intended to be limiting.

As used in the specification and claims, the singular forms “a”, “an” and “the” include plural references unless the context clearly indicates otherwise.

Unless defined otherwise, all technical and scientific terms used herein have the same meaning as commonly understood by one of the ordinary skill in the art to which the invention belongs.

The term “treating”, “treat” or “treatment” as used herein includes preventive (prophylactic) treatment.

The term “treating”, “treat” or “treatment” as used herein includes palliative treatment. The term “Indigofera heterantha” also includes the synonyms such as “Indigofera gerardiana”.

“Extract” or “isolated extract” mentioned here means a blend of compounds present in or obtained from the plant Indigofera heterantha. Such a blend of compounds is obtained by extraction of the whole plant or parts of the plant Indigofera heterantha such as roots, twigs, stem, leaves and inflorescence using solvents optionally followed by further enrichment.

“Herbal composition” mentioned herein refers to a composition comprising a therapeutically effective amount of extract of the plant Indigofera heterantha, either alone or in combination with a pharmaceutically acceptable carrier.

As used herein, the term “therapeutically effective amount” means an amount of the extract of the plant Indigofera heterantha effective in preventing infection by the virus and/or an amount of the extract of the plant Indigofera heterantha that yields a desired therapeutic response such as, alleviating, treating and/or preventing the symptoms of skin lesions, sores, cold sores, blisters, warts, lumps, bumps, pimples, rashes and ulcers associated with or caused by a viral infection.

By “pharmaceutically acceptable” it is meant the carrier, diluent, excipients, and/or salt must be compatible with the other ingredients of the formulation, and not deleterious to the recipient thereof.

As used herein, the term “pharmaceutically acceptable carrier” means a non-toxic, inert solid, semi-solid, diluent, encapsulating material or formulation auxiliary of any type. Some non-limiting examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; malt; gelatin; talc; as well as other non-toxic compatible lubricants such as sodium lauryl sulfate and magnesium stearate; as well as coloring agents; releasing agents; coating agents; sweetening, flavoring and perfuming agents; preservatives such as phenolip, methyl paraben and butyl paraben; antioxidants; oils or waxes such as beeswax, carmauba wax, hard wax, yellow wax and cetyl esters; emulsifiers; petrolatums such as paraffin, lanolin alcohols, white petrolatum, yellow petrolatum, wool alcohols, petroleum jelly and petroleum wax; glycols such as propylene glycol, methyl glycol and methyl ethylene glycol; carbomers such as carpopol 974P; poly oxy ethylene alkyl ethers such as cetosteryl alcohol; plasticizers such as triethanolamine; solvents and hydrophilic gelling agents can also be present in the composition, according to the judgment of the formulator.

The plant Indigofera heterantha Wallich ex Brandis is a commonly distributed species in the Western Himalayas in India. The whole plant of the species or parts of the plant such as roots, twigs, stem, leaves and inflorescence were collected from the hills of Uttarakhand, India. The freshly collected plants or parts of the plant were dried. For taxonomic characterization, herbarium specimens in flowering and fruiting were collected and deposited in the departmental herbarium of Piramal Life Sciences Limited, Mumbai, India. Based on morphological characters, the specimen was identified as Indigofera gerardiana Wallich ex Baker, which is now a synonym for the taxonomically valid species Indigofera heterantha Wallich ex Brandis (Fascicles of Flora of India, Fascicle 21, Leguminosae—Papilionoideae: Tribe—Indigofereae, 1995, 76-79). The extracts obtained and used in this invention are not limited to those obtained from Indigofera heterantha plants grown in the Western Himalayas and the extract may be obtained from any Indigofera heterantha plant.

The present invention relates to an isolated extract from whole plant or one or more parts of the plant Indigofera heterantha prepared by stirring in a solvent; concentrating the extract; and optionally enriching the extract by solvent partitioning or chromatography.

The present invention further relates to a herbal composition comprising a therapeutically effective amount of an extract of whole plant or one or more parts of the plant Indigofera heterantha prepared by stirring in a solvent; concentrating the extract; and optionally enriching the extract by solvent partitioning or chromatography; either alone or in combination with a pharmaceutically acceptable carrier.

The invention also relates to the process for the preparation of the herbal composition comprising extract of the plant Indigofera heterantha. The process includes the following steps:
(a) preparing an extract from the whole plant or one or more parts of the plant Indigofera heterantha by stirring in a solvent in a ratio of 1:5 to 1:40 weight/volume for 2 hours to 24 hours at 40° C. to 50° C.;
(b) concentrating the solvent extract obtained in step (a);
(c) optionally drying the extract obtained in step (b) under high vacuum (0.01-5 mm Hg);
(d) optionally enriching the extract obtained in step (b) or step (c) using materials selected from polyamide resin, gelatin/sodium chloride solution, polyvinylpyrrolidone, caffeine, lead (II) acetate or hide powder;
(e) optionally enriching the extract obtained in step (b), step (c) or step (d) by solvent partitioning or chromatography; and
(f) optionally mixing the extract of step (b), step (c), step (d) or step(e) with a pharmaceutically acceptable carrier.

In an aspect of the invention, the process for the preparation of the herbal composition comprising extract of the roots of the plant Indigofera heterantha includes the following steps:
(a) preparing an extract from the roots of the plant Indigofera heterantha by stirring in a solvent in a ratio of 1:5 to 1:40 weight/volume for 2 hours to 24 hours at 40° C. to 50° C.;
(b) concentrating the solvent extract obtained in step (a);
(c) optionally drying the extract obtained in step (b) under high vacuum (0.01-5 mm Hg)
(d) optionally enriching the extract obtained in step (b) or step (c) using materials selected from polyamide resin, gelatin/sodium chloride solution, polyvinylpyrrolidone, caffeine, lead (II) acetate or hide powder;
(e) optionally enriching the extract obtained in step (b), step (c) or step (d) by solvent partitioning or chromatography; and
(f) optionally mixing the extract obtained in step (b), step (c), step (d) or step (e) with a pharmaceutically acceptable carrier.

In an aspect of the invention, the solvent for extracting whole plant or one or more parts of the plant Indigofera heterantha is selected from methanol, ethanol, n-propanol, isopropanol, n-butanol, acetone, ethyl acetate, dichloromethane, water, or mixtures thereof, preferably mixture of methanol and water or ethanol and water.

In another aspect of the invention, the solvent extract is filtered before concentration.

In another aspect of the invention, concentration of the solvent extract is done by using one or more of the methods selected from (i) distillation under reduced pressure (150-600 mm Hg) at 30° C. to 50° C.; (ii) lyophilization; and (iii) spray drying to obtain the extract.

In yet another aspect of the invention, the extract is enriched using materials such as polyamide resin, gelatin/sodium chloride solution, polyvinylpyrrolidone, caffeine, lead (II) acetate or hide powder (collagen material obtained from hides), preferably polyamide resin, in a 1:3 to 1:5 ratio of extract to polyamide resin.

In another aspect of the invention, the solvents for enriching the extract by solvent partitioning are selected from water, petroleum ether, dichloromethane, chloroform, ethyl acetate, methanol, acetone, acetonitrile, n-propanol, iso-propanol, and butanol or mixtures thereof.

In another aspect of the invention, the enrichment of the extract by chromatography can be done by one or more of the following methods: normal phase chromatography (using alumina or silica gel); reverse phase chromatography (using reverse phase silica gel such as dimethyloctadecylsilyl silica gel, (RP-18) or dimethyloctylsilyl silica gel (RP-8); gel permeation chromatography (using resins such as Sephadex LH-20® (Pharmacia Chemical Industries, Sweden), or Sephadex® G-10 and G-25); or by counter-current chromatography (using a biphasic eluent system). These techniques may be used repeatedly, alone or in combination.

The present invention further relates to a method for treating viral infection, which comprises administering to a mammal in need thereof, the herbal composition comprising extract of the plant Indigofera heterantha.

The present invention further relates to a method for treating viral infection, particularly for the treatment of viral infection caused by HSV, more particularly HSV-2, which comprises administering to a mammal in need thereof, the herbal composition comprising extract of the plant Indigofera heterantha.

The present invention also relates to the use of the herbal composition comprising extract of the plant Indigofera heterantha, for the treatment of viral infection, particularly for the treatment of viral infection caused by HSV, more particularly HSV-2.

The present invention also relates to the use of the extract of the plant Indigofera heterantha for the manufacture of a medicament for the treatment of viral infection, more particularly for the treatment of viral infection caused by HSV.

In an aspect of the invention, the mammal to be treated or the mammal to which the use is directed is a human who has been diagnosed as having an infection caused by a virus. More particularly, the mammal to be treated is a human who has been diagnosed as having an infection caused by a HSV.

In another aspect of the invention, the mammal to be treated is a human who has been diagnosed as being infected with human immunodeficiency virus (HIV) to whom the herbal composition is administered as a prophylactic measure against co-infection with HSV-2.

In yet another aspect of the invention, the mammal to be treated is a human to whom the herbal composition is administered as a prophylactic measure against sexually transmitted infection (STI).

In an aspect of the invention, the method for treating viral infection includes the administration of herbal composition described above, by known administration routes, modes, etc. including the following:

The herbal composition can be administered orally, for example in the form of pills, tablets, coated tablets, capsules, granules, solutions, elixirs or syrup.

The extract of the plant Indigofera heterantha is used to prepare oral preparations containing about 5 to about 99% by weight of the extract, which is blended into a conventional base.

The herbal composition can be used for topical or transdermal administration. The compositions useful in the present invention involve formulations suitable for topical or transdermal application to skin, administration to mucous membranes, or administration in conjunction with a condom or other barrier device. The compositions can be formulated into a wide variety of product types that include but are not limited to lotions, creams, gels, sticks, patches, vaginal suppositories or pessaries, sprays or ointments.

The extract of the plant Indigofera heterantha is used to prepare topical or transdermal preparations containing about 5 to about 99% by weight of the extract, preferably 5 to 50%, which is blended into a conventional base.

The extract of the plant Indigofera heterantha is present in the herbal composition of the present invention in such an amount which is effective to achieve the desired therapeutic response for a particular patient without being toxic to the patient or causing severe side effects. The selected amount will depend upon a variety of factors including the activity of the extract of the present invention employed, the route of administration, the time of administration, the rate of excretion of the particular composition being employed, the duration of the treatment, the age, sex, weight, condition, general health and prior medical history of the patient being treated, and like factors well known in the medical arts.

The efficacy of the extract of the plant Indigofera heterantha has been established by biological assays which are described in detail in subsequent examples. These examples are herein provided for the purpose of illustration only and are not intended to limit the scope of the invention.

### EXAMPLES

### Example 1

Preparation of Methanol Extract of the Roots of the Plant Indigofera heterantha.

The freshly collected roots of Indigofera heterantha were dried (350 g) and pulverized. The coarsely ground material was soaked in 3.5 L methanol, with constant stirring, for 8 hours in a stainless steel vessel that was placed in the water bath maintained at 45° C. The extract was filtered and the residue was soaked in 3.5L of methanol for 16 hours at room temperature and filtered. The extracts were combined and concentrated using rotary evaporator for 5 hours at 45° C. under line vacuum (about 500 mm Hg), and dried using the Speed Vac® Plus (Savant, USA) for 8 hours at approximately 40° C. to 50° C. to obtain 25.78 g of the extract.

The extract is dark brown in color and partially soluble in water.

### Example 2

Preparation of Water Extract of the Roots of the Plant Indigofera heterantha.

The freshly collected roots of Indigofera heterantha were dried (100 g) and pulverized. 1000 mL of water was added to the pulverized roots in a 2 L conical flask and was constantly stirred, using a magnetic stirrer, over a hot plate for 3 hours at 45° C. The extract was filtered under line vacuum (about 500 mm Hg). The filtrate (800 mL) was lyophilized using Freeze Dryer (Edwards, Italy) for 8 hours to obtain 6.5 g of the extract.

The extract is brown in color.

### Example 3

### Enrichment of the Extract of Example 1.

Extract (112 mg) of Example 1 was dissolved in 8 mL of methanol with the help of vortex stirrer and sonicator. 300 mg of polyamide 6 resin (Macherey Nagel, Germany) was added to the extract and vortexed; allowed to stand for 45 minutes and filtered. The filtrate was dried using Speed Vac® Plus (Savant, USA) for 12 hours to obtain 54.6 mg of the extract.

The extract is golden brown in color and partially soluble in water.

### Example 4

### Enrichment of the Extract of Example 3.

Extract of Example 3 (5 g; prepared according to Example 3) was suspended in 100 mL of water:methanol (9:1) mixture, at room temperature (25° C.) and sonicated and partitioned three times successively with 300 mL (100 mL×3) petroleum ether.

The aqueous layer obtained from the above step was partitioned three times successively with 300 mL (100 mL×3) chloroform.

The aqueous layer obtained from the above step was partitioned three times successively with 300 mL (100 mL×3) ethyl acetate.

The final aqueous layer obtained from the above step was concentrated in a rotary evaporator under line vacuum (about 500 mm Hg) followed by lyophilization to obtain 1.19 g of light brown-colored extract.

### Example 5

Preparation of Hydro-Alcoholic (Methanol:Water) Extract of the Roots of the Plant Indigofera heterantha.

The roots of Indigofera heterantha were dried (200 g), pulverized and soaked in 1.6L methanol:water (1:1), with constant stirring, for 3 hours in a water bath maintained at 40° C. ±5° C. The extract was filtered and the residue was soaked in 1.6 L of methanol:water (1:1) and the same process was repeated two more times. The extracts were combined and concentrated using rotary evaporator at 45° C. under line vacuum (about 500 mm Hg), and dried using the Speed Vac® Plus (Savant, USA) to obtain 28.97 g of the extract.

The extract is dark brown in color.

### Example 6

Preparation of Hydro-Alcoholic (Ethanol:Water) Extract of the Roots of the Plant Indigofera heterantha.

The roots of Indigofera heterantha were dried (200 g) and pulverized and soaked in 1.6 L ethanol:water (1:1), with constant stirring, for 3 hours in a water bath maintained at 40° C. ±5° C. The extract was filtered and the residue was soaked in 1.6 L of ethanol:water (1:1) and the same process was repeated two more times. The extracts were combined and concentrated using rotary evaporator at 45° C. under line vacuum (about 500 mm Hg), and dried using the Speed Vac® Plus (Savant, USA) to obtain 29.89 g of the extract.

The extract is dark brown in color.

### Example 7

### Enrichment of Extract of Example 1

(a) Extract (150 g) of Example 1 was suspended in 2 L of water at room temperature (25° C.) and sonicated. The extract was filtered to obtain an aqueous filtrate. The residue (37.05 g) was lyophilized using Freeze Dryer (Edwards, Italy).
(b) The aqueous filtrate collected in step (a) was partitioned three times successively with 300 mL (100 mL×3) chloroform to obtain an aqueous layer and a chloroform layer. The chloroform layer was concentrated under reduced pressure at 45° C. to obtain 0.425 g of chloroform fraction.
(c) The aqueous layer obtained from step (b) was partitioned three times successively with 300 mL (100 mL×3) ethyl acetate to obtain an aqueous layer and an ethyl acetate layer. The ethyl acetate layer was concentrated under reduced pressure at 45° C. to obtain 3.737 g of ethyl acetate fraction.
(d) The aqueous layer obtained from step (c) was concentrated under reduced pressure and lyophilized to obtain 89 g of aqueous fraction [Example 7 (d)].
(e) 1 g of dried aqueous fraction obtained from step (d) was refluxed for 1 h with 50 mL ethyl acetate and filtered. 0.053 g of ethyl acetate filtrate and 0.853 g of ethyl acetate residue [Example 7 (e)] were obtained after concentration.

### Example 8

Preparation of Formulation of Extract of Example 5, Extract of Example 6 and Extract of Example 7 (d) from the Roots of the Plant Indigofera heterantha.

General Procedure for the Preparation of Cream.

Required amount of ingredient 6 (refer to Table 1, Table 2 and Table 3) was added in a suitable glass/stainless steel vessel. Ingredient 1 was added to the vessel and dissolved/dispersed using mechanical stirrer. The temperature was maintained at 60° C. to 75° C. Ingredients 4 and 5 were added to this solution under constant stirring. Ingredients 2 and 3 were melted and added to the above vessel under constant stirring. The temperature was reduced slowly to room temperature (25° C.).

TABLE 1
Formulation I

| | | IA | IB | IC | ID |
|---|---|---|---|---|---|
| No. | Ingredients | % w/w | % w/w | % w/w | % w/w |
| 1 | Extract of Example 5 | 5 | 10 | 20 | 40 |
| 2 | Beeswax | 2 | 2 | 2 | 2 |
| 3 | Paraffin | 74.09 | 69.09 | 59.09 | 40.75 |
| 4 | Propylene glycol | 2 | 2 | 2 | 2 |
| 5 | Phenolip | 0.25 | 0.25 | 0.25 | 0.25 |
| 6 | Water | 16.66 | 16.66 | 16.66 | 15 |
| | Total | 100 | 100 | 100 | 100 |

TABLE 2
Formulation II

| | | IIA | IIB | IIC | IID |
|---|---|---|---|---|---|
| No. | Ingredients | % w/w | % w/w | % w/w | % w/w |
| 1 | Extract of example 6 | 5 | 10 | 20 | 40 |
| 2 | Beeswax | 2 | 2 | 2 | 2 |
| 3 | Paraffin | 74.09 | 69.09 | 59.09 | 40.75 |
| 4 | Propylene glycol | 2 | 2 | 2 | 2 |
| 5 | Phenolip | 0.25 | 0.25 | 0.25 | 0.25 |
| 6 | Water | 16.66 | 16.66 | 16.66 | 15 |
| | Total | 100 | 100 | 100 | 100 |

TABLE 3
Formulation III

| | | IIIA | IIIB | IIIC | IIID |
|---|---|---|---|---|---|
| No. | Ingredients | % w/w | % w/w | % w/w | % w/w |
| 1 | Extract of Example 7 (d) | 5 | 10 | 20 | 40 |
| 2 | Beeswax | 2 | 2 | 2 | 2 |
| 3 | Paraffin | 74.09 | 69.09 | 59.09 | 40.75 |
| 4 | Propylene glycol | 2 | 2 | 2 | 2 |
| 5 | Phenolip | 0.25 | 0.25 | 0.25 | 0.25 |
| 6 | Water | 16.66 | 16.66 | 16.66 | 15 |
| | Total | 100 | 100 | 100 | 100 |

### Example 9

### Preparation of Formulation of Extract of Example 1.

General Procedure for the Preparation of Gel.

Weighed amount of ingredient 2 (refer to Table 4) was dissolved in warm water. Ingredient 1 was added to the vessel and dissolved/dispersed using mechanical stirrer. The solution was cooled to room temperature and few drops of ingredient 3 were added under stirring to obtain a clear gel.

TABLE 4
Formulation IV

| No. | Ingredients | % w/w |
|---|---|---|
| 1 | Extract of Example 1 | 50 |
| 2 | Carpopol 974P | 1 |
| 3 | Triethanolamine | qs |
| 4 | Water | 49 |
| | Total | 100 |

qs: quantity sufficient

### Biological Evaluation

### In Vitro Antiviral Assays

### Example 10

### Preparation of Viral Stock.

Materials used:

| | |
|---|---|
| Cell line | Vero (Kidney epithelial cells of African green |
| | monkey kidney cell line-American Type |
| | Culture Collection (ATCC) # CCL-81) |
| Virus | HSV-2 (ATCC strain VR-734 and clinical strain |
| | no. 753167 from National Institute of Virology, |
| | Pune, India) |
| Medium | Dulbecco's Modified Eagle Medium |
| | (DMEM, Gibco, USA, Cat no: 12430) |
| Serum | Fetal Bovine Serum |
| | (FBS, Gibco, USA, Cat no: 16000-044) |
| Trypsin-EDTA solution | 0.25% Trypsin-Ethylenediaminetetra-aceticacid |
| | (Trypsin-EDTA, Gibco, USA, Cat no: 25200) |
| Standard compound | Acyclovir (Medicorp, Hyderabad, India) |
| Plasticwares | Tissue culture flasks 25 cm² |
| | (Nunc, USA, Cat no: 156367) |
| | Tissue culture flasks 75 cm² |
| | (Nunc, USA, Cat no: 156499) |
| | Centrifuge tubes 15 mL |
| | (Nunc, USA, Cat no: 366060) |
| | Centrifuge tubes 50 mL |
| | (Nunc, USA, Cat no: 373687) |
| | Flat bottom 96-well plates |
| | (Nunc, USA, Cat no: 167008) |
| Stain | Crystal violet (Sigma, USA, Cat no: |
| | C3886-25G) |

Antibiotic-antimycotic mixture (Gibco, USA, Cat no: 15240)

### Step 1

### Maintenance of the Cell Line.

Maintenance of the cell line was performed as reported in Antiviral Research, 2005, 67, 24-30.

Vero cell line obtained from ATCC was initiated from the kidney of a normal adult African green monkey. This cell line was propagated in complete growth medium i.e. Dulbecco's Modified Eagle Medium (DMEM) supplemented with 10% Fetal Bovine Serum (FBS) and 1×antibiotic-antimycotic mixture. T-25 tissue culture flask with cell monolayer was selected for subculturing. DMEM from the flask was removed and briefly rinsed with DMEM without serum to remove all traces of serum that contains trypsin inhibitor. 1 mL of Trypsin-EDTA solution was added to flask and observed under an inverted microscope until cell monolayer was dispersed (usually within 3-5 minutes). Immediately, 14 mL of complete growth medium was added and cells were aspirated by gentle pipetting. A subcultivation ratio of 1:3 was obtained by adding each 5 mL of cell suspension to 3 different T-25 tissue culture flasks. Flasks were maintained at 37° C. with 5% CO₂.

### Step 2

### Virus (HSV-2) Propagation.

Virus propagation was performed as reported in Antiviral Research, 2005, 67, 24-30. Commercially available strain of HSV-2, derived from a human with the genital infection, was obtained from ATCC (ATCC VR-734, virus titer 10^{5.75} TCID₅₀/0.2 mL). For HSV-2 propagation Vero cell line was used as target cells. T-75 tissue culture flask with 24-48 hours old and 80-90% confluent monolayer of Vero cells (obtained in step 1) was selected for virus infection. On the day of infection, Vero cells were inoculated with 1 mL of HSV-2 inoculum at original titer equivalent to 10^{3.1} TCID₅₀/0.2 mL and incubated for 30 minutes at 37° C. for virus adsorption. Post incubation, 10 mL of maintenance medium (DMEM with 2% FBS) was added to the flask and incubated at 37° C. with 5% CO₂ for 48 hours till complete disruption of cell monolayer. Flask was observed microscopically twice daily for cytopathic effect (CPE). CPE are alterations in cellular morphology, such as rounding and enlargement of cells, synctia and inclusion formation, caused by the virus. After 48 hours of incubation, the flask was given 2-3 freeze thaw cycles for complete disruption of cells and release of the virus into the culture medium. Cell debris were removed by centrifugation (1000 rpm, 10 minutes, 4° C.) and the supernatant, which represents the viral stock, was stored in aliquots at −80 ° C. Titer of the viral stock was determined using following methods:

### Step 3 (A)

### Determination of Viral Titer Using CPE Assay.

The assay was done as reported in World J. Gastroenterol., 2006, 12: 4078-4081.

Viral titer was determined by CPE assay and was expressed as tissue culture infectious dose 50 (TCID₅₀). Vero cells (obtained in step 1) were seeded in 96-well plate at a density of 2×10⁴ cells/100 μL/well and then incubated at 37° C. with 5% CO₂ for 24 hours for 80-90% confluency. A serial dilution of viral stock (obtained in step 2) was carried out (10⁻¹ to 10⁻⁸) in maintenance medium (DMEM with 2% FBS). Growth medium from the culture plate was removed and 100 μL of each dilution of virus was used for infecting Vero cells. Vero cells only with maintenance medium served as cell control. Post infection, the culture plate was incubated at 37° C. in a CO₂ incubator for 48 hours. After 48 hours of incubation, the CPE was examined under an inverted microscope in the wells inoculated with virus dilutions. When virus controls showed the maximum CPE, media was removed and the infected monolayer was fixed and stained using a solution containing formalin (10%) and crystal violet (1%) for 30 minutes. At the end of 30 minutes, the stain was aspirated out and the plate rinsed using distilled water until all excess stain was washed away. The plate was allowed to dry overnight. The viral titer (TCID₅₀) was calculated as described in Am. J. Hyg., 1938, 27, 493-497. TCID₅₀ represents the dose that gives rise to CPE in 50% of inoculated cultures.

Result: The TCID₅₀ value for this experiment was 3.98×10⁶.

### Step 3 (B)

### Determination of Viral Titer Using Plaque Assay.

The assay was done as reported in Antiviral Res., 2005, 67(1): 24-30.

Viral titer was also determined by plaque assay and was expressed as plaque forming units per mL (pfu/mL). Vero cells (obtained in step 1) were trypsinized, counted and plated into 24-well plate at a density of 2×10⁵ cells/mL/well and incubated at 37° C. with 5% CO₂ for 24 hours for 80-90% confluency. Serial dilutions of virus (from viral stock obtained in step 2) were prepared in the range of 10⁻² to 10⁻⁷ using maintenance medium (DMEM with 2% FBS). Growth medium from the plate was removed and 0.2 mL of each dilution of virus was added to each well taking care not to dislodge any cells. Infected monolayers were incubated at 37° C. with 5% CO₂ for 1 hour with shaking every 15 minutes. After the incubation period, 1% CMC was added to each well in 1 mL volume and plate was incubated for 48 hours, after which the cells were fixed and stained with a solution containing formalin (10%) and crystal violet (1%) for 30 minutes. At the end of 30 minutes, the stain was aspirated out and the plate was rinsed using distilled water until all excess stain was washed away. The plates were allowed to dry overnight. Plaques were counted to estimate the viral titer which is expressed as plaque forming units per mL (pfu/mL).

Viral titer=(No. of plaques produced×dilution of virus×vol. of inoculum)

Result: Viral titer determined by the plaque assay was 1.4×10⁷ pfu/mL.

### Example 11

Primary Antiviral Screening Test was Performed using CPE Inhibition Assays.

Method A: CPE inhibition assay—Crystal violet staining method.

The assay was designed to detect agents (in this case, the extracts) acting at any stage of the virus reproductive cycle. The assay was done as reported in Indian J. Med. Res., 2004, 120:24-29.

Vero cells (obtained in step 1 of Example 10) were propagated at a density of 1×10⁴ cells/well in 96 well plate and incubated at 37° C. in a CO₂ incubator for 24 hours to form a monolayer. Extract of Example 1, extract of Example 2, extract of Example 3, extract of Example 5 and extract of Example 7 (e) were tested by adding at either at 50 μg/mL or 100 μg/mL concentration or both (DMSO stock of 20 mg/mL of the extract was diluted to 50 μg/mL or 100 μg/mL with DMEM containing 2% FBS) in a final culture volume of 200 μL/well. Appropriate controls were included such as Vero cells alone (cell control), Vero cells with virus (virus control) and Vero cells with virus and the standard compound acyclovir. Acyclovir was checked at the following concentrations (DMSO stock of 20 mg/mL of acyclovir was diluted to 100 μg/mL with DMEM containing 2% FBS): 25 μg/mL, 3.125 μg/mL, 1.56 μg/mL and 0.78 μg/mL and 0.39 μg/mL. After one hour, cells were infected with a multiplicity of infection (moi) of 100 TCID₅₀ of viral dose per well using viral stock obtained in step 2 of Example 10. The infected cells were incubated with maintenance medium (DMEM with 2% FBS) for another 48 hours. When virus controls showed the maximum CPE, medium was aspirated and the cells were stained with 1% crystal violet solution for 30 minutes. The staining solution was aspirated out and the plates rinsed using distilled water until all excess stain was washed away. The plates were allowed to dry for 24 hours. CPE was evaluated visually, after staining the plaques, and microscopically and graded according to the percentage of CPE inhibition as compared to controls. Results obtained are given in Table 5.

TABLE 5

| Sample | Concentration | CPE inhibition |
|---|---|---|
| Extract of Example 1 | 100 μg/mL | ++++ |
| Extract of Example 2 | 100 μg/mL | ++++ |
| Extract of Example 3 | 100 μg/mL | ++++ |
| Extract of Example 5 | 50 μg/mL | ++++ |
| Extract of Example 7 (e) | 50 μg/mL | +++ |
| | 100 μg/mL | ++++ |
| Acyclovir | 25 μg/mL | ++++ |

Grading is according to the following format:
+11-25% CPE inhibition; ++26-50% CPE inhibition;
+++51-75% CPE inhibition; ++++76-100% CPE inhibition.

Method B: CPE Inhibition Assay—MTT Method.

The assay was designed to detect agents (in this case, the extracts) acting at any stage of the virus reproductive cycle. The assay was done as reported in World J. Gastroenterol., 2006, 12:4078-4081.

This assay was performed as described in Method A of Example 11, for CPE inhibition assay-staining method, except that 3-(4,5-dimethylthiazol-2yl)-2,5-diphenyltetrazolium bromide (MTT) assay was carried out without staining of the cells with crystal violet staining. Briefly, Vero cells (obtained in step 1 of example 10) in 96-well flat-bottomed plates were treated with maintenance medium (DMEM with 2% FBS) that contained extract of Example 1, extract of Example 3 or acyclovir for 1 hour. Then cells were infected with virus (using viral stock obtained in step 2 of example 10) at a moi of 100 TCID₅₀. After 48 hour incubation at 37° C., viable cells were measured (absorbance at 570 nm was measured by using 96-well plate ELISA reader) by MTT assay. The antiviral activity was determined according to the following formula:

$$Antiviral activity = \frac{{( {OD}_{T} )}_{HSV} - {( {OD}_{C} )}_{HSV}}{{( {OD}_{C} )}_{mock} - {( {OD}_{C} )}_{HSV}} \times 100$$

wherein:
(OD_{T})_{HSV}: absorbance measured with a concentration of extract/compound in HSV infected cells;
(OD_{C})_{HSV}: refers to absorbance measured for the control untreated HSV-infected cells; and
(OD_{C})_{mock}: refers to absorbance measured for control untreated mock-infected cells.

Result: The extract of Example 1 and extract of Example 3 exhibited antiviral activity against HSV-2.

### Example 12

### CPE Inhibition Assay—MTT Method.

This assay was performed as mentioned in Method B of Example 11.

The extracts evaluated in this assay were:
(i) Extract of Example 2
(ii) Extract of Example 5
(iii) Extract of Example 6
(iv) Extract of Example 7 (d)

Samples were tested in the concentration range of 6.25 μg/mL to 400 μg/mL.

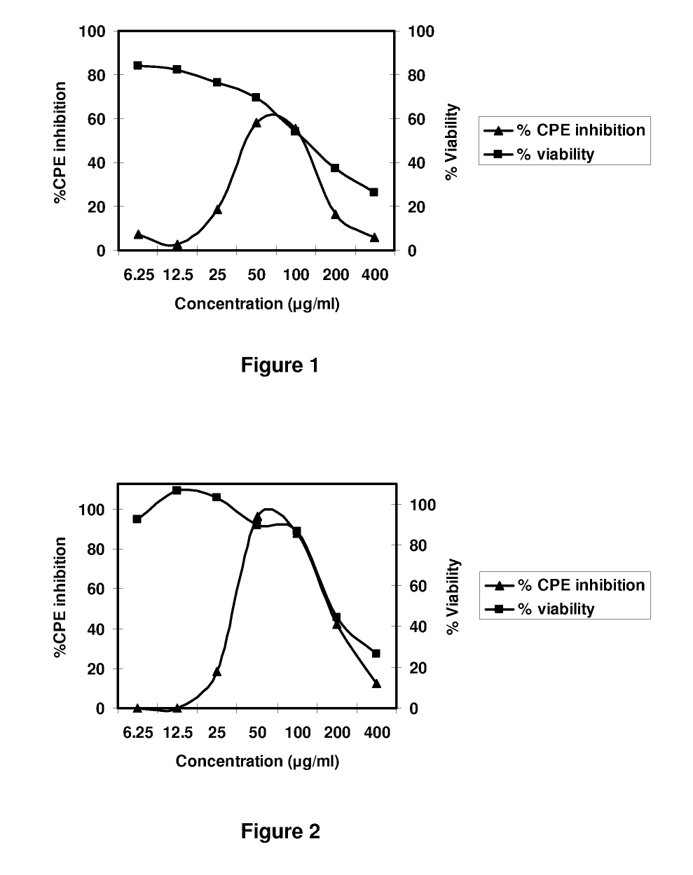

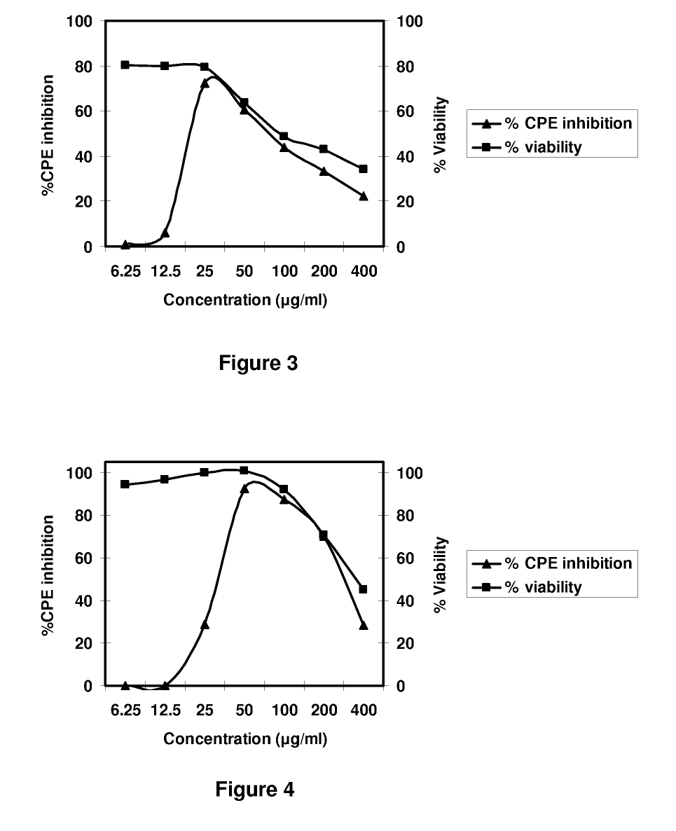

Results: The results are depicted in FIG. 1, FIG. 2, FIG. 3 and FIG. 4 for extract of Example 2, extract of Example 5, extract of Example 6 and extract of Example 7 (d) respectively.

Conclusions:

FIG. 1: Extract of Example 2 exhibited antiviral activity against HSV-2. The extract of Example 2 did not affect the viability of Vero cells at concentrations exhibiting antiviral activity.

FIG. 2: Extract of Example 5 exhibited antiviral activity against HSV-2. The extract of Example 5 did not affect the viability of Vero cells at concentrations exhibiting antiviral activity.

FIG. 3: Extract of Example 6 exhibited antiviral activity against HSV-2. The extract of Example 6 did not affect the viability of Vero cells at concentrations exhibiting antiviral activity.

FIG. 4: Extract of Example 7 (d) exhibited antiviral activity against HSV-2. The extract of Example7 (d) did not affect the viability of Vero cells at concentrations exhibiting antiviral activity.

### Example 13

### Cytotoxicity Assay.

The assay was done as reported in World J. Gastroenterol., 2006, 12:4078-4081.

Toxicity analysis was performed in order to assess whether any observed antiviral effects resulted from a general effect on cell viability. Vero cells (obtained in step 1 of example 10) for the toxicity analyses were cultured in 96-well plates and treated with extracts with the same schedule as used for antiviral evaluations without addition of virus. Viable cells were assayed using the MTT dye. Toxic effects of extract of Example 3 were calculated as a percentage of the reduction of viable cells in the presence of each plant extract as compared to viable cells observed in the absence of plant extract. The following formula was used:

$$Cytotoxicity = \frac{\{ A ( compound ) - A ( Blank ) \}}{\{ ( A ( Cell control ) - A ( Blank ) \}} \times 100$$

wherein A represents absorbance measured at ELISA reader.

The 50% cell cytotoxic concentration (CC₅₀) was calculated from this data.

The selectivity index (SI), also referred to as therapeutic index, was evaluated as the ratio of CC₅₀ and IC₅₀ and the results obtained are given in Table 6. To determine if the extract of Example 3 has sufficient antiviral activity that exceeds its level of toxicity, SI was calculated according to CC₅₀/IC₅₀.

For present study SI value of >5 has been considered as effective for herbal/plant extracts.

TABLE 6

| | *IC₅₀ | CC₅₀ | SI |
|---|---|---|---|
| Sample | (μg/mL) | (μg/ml) | (CC₅₀/IC₅₀) |
| Extract of Example 3 | 75 | >400 | >5.33 |

*IC₅₀ value obtained from Example 11 Method B.

### In Vivo Antiviral Assays

Animals used in the experiments were housed and cared for, in accordance with the

Guidelines in force published by CPCSEA (Committee for the Purpose of Control and Supervision of Experiments on Animals), Tamil Nadu, India. Procedures using laboratory animals were approved by the IAEC (Institutional Animal Ethics Committee) of Piramal Life Sciences Limited, Goregaon, Mumbai, India.

### Example 14

### Mouse Vaginal Model of HSV-2 Infection.

The assay was done as reported in Antiviral Research, 2006, 69:77-85.

Female BALB/c mice of eight weeks age and body weight 18-20 g were used for intravaginal (IVAG) challenge (vaginal inoculation) with the virus (using viral stock of step 2 of example 10). Five days prior to the IVAG challenge, mice were injected subcutaneously (SC) with 2 mg of progesterone (Depo-Provera®; Pfizer, Belgium) in the upper back, using a 29-gauge needle. On the day of challenge, mice were anesthetized with Ketamine (150 mg/kg) and Xylazine (10 mg/kg) by intraperitoneal injection. Mice were inoculated intravaginally with 5×10³ pfu of the virus using a micropipette in a total volume of 20 μL DMEM. Treatment of animals was started 30 minutes after the IVAG challenge with placebo (Phosphate Buffered Saline, PBS), extract of Example 3 (125 mg/kg in PBS), extract of Example 4 (300 mg/kg in PBS) and the positive control (75 mg/kg acyclovir in PBS). Each group had ten animals. Animals were treated three times a day with the treatment intervals of 4 hours for 5 days. 20 μL of the above samples were injected into the vaginal vault using micropipette.

The animals were assessed daily for survival and extravaginal disease signs through 21 days of post inoculation (PI). The severity of the viral disease (extravaginal signs of disease) was quantified using a well-established lesion score scale, also known as five-point scale as follows:
0: no apparent infection;
1: few isolated papules and slight redness of extravaginal tissue;
2: few isolated papules, ulcers, and/or eschar and/or swelling and redness of extravaginal tissue;
3: multiple fused ulcers/eschars, moderate swelling and redness of extravaginal tissue with extension to surrounding tissue;
4: ulceration with severe redness and swelling of extravaginal tissue with extension to surrounding tissue, rear leg paralysis; and
5: severe ulceration of extravaginal tissue with extension to surrounding tissue, weight loss, rear leg paralysis and death.

Observations:
1. Group treated with placebo:
   (a) The earliest sign of extravaginal infection occurred on day 5.
   (b) All the mice died by day 11.
2. Group treated with Acyclovir:
   (a) None of the mice showed signs of extravaginal disease.
   (b) None of the mice died during the experiment.
3. Group treated with extract of Example 3:
   (a) Mice treated with 125 mg/kg of the extract of Example 3 did not exhibit any characteristic signs of virus-induced extravaginal disease at any time during the experiment.
   (b) Mice treated with 125 mg/kg of extract of Example 3 showed 100% survival rate.
4. Group treated with extract of Example 4:
   (a) Nine mice out of ten treated with 300 mg/kg extract of Example 4 did not exhibit any characteristic signs of virus-induced extravaginal disease at any time during the experiment. One mouse exhibited characteristic signs of HSV-induced extravaginal disease on day 5. However, this mouse clinically recovered by day 19.
   (b) Mice treated with 300 mg/kg extract of Example 4 showed 100% survival rate.

Result: The extract of Example 3 and extract of Example 4 exhibited antiviral activity in the mouse vaginal model of HSV-2 infection.

### Example 15

### Mouse Vaginal Model of HSV-2 Infection.

The assay was performed as mentioned in Example 14 with modifications as mentioned below:
(i) Mice were inoculated intravaginally with 2×10⁴ pfu of the virus instead of 5×10³ pfu in Example 14.
(ii) Evaluation was done by topical application of the formulations (Formulation I, Formulation II and Formulation III) instead of injection of extracts into vaginal vault of the animals in Example 14.
(iii) One more group of animals was studied: “infection control group” in which the mice were inoculated with the virus but were not treated. This group was used to monitor the course of the disease and to compare it with the “placebo treated group” if placebo shows any effect on the diseased animals.
(iv) Dose of Acyclovir evaluated was 225mg/kg body weight instead of 75 mg/kg in Example 14.

The following extracts and formulations have been evaluated:
(a) Extract of Example 1
(b) Formulation I
(c) Formulation II
(d) Formulation III

Sample application to the animals:

Extract of Example 1 (dissolved in PBS) was injected into the vaginal vault using micropipette. Formulation I, Formulation II and Formulation III were applied topically (25 mg) thrice daily for a 5 day period. 25 mg of Formulation IB, IIB or IIIB correspond to 375 mg/kg dose; 25 mg of Formulation IC, IIC or IIIC correspond to 750 mg/kg dose and 25 mg of Formulation ID, IID and IIID correspond to 1500 mg/kg dose evaluated in the animals.

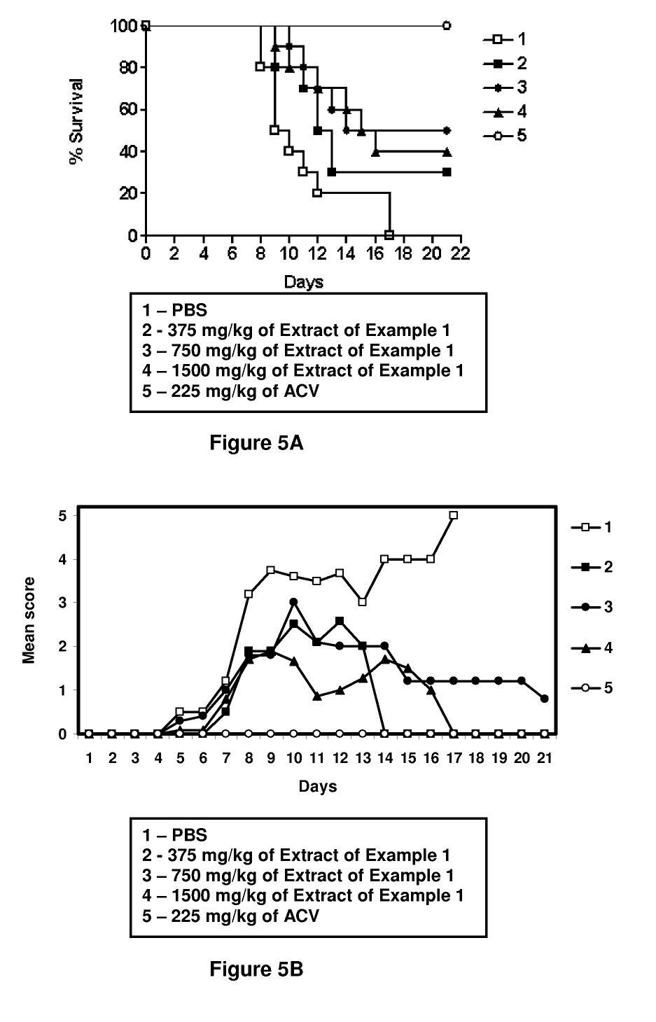

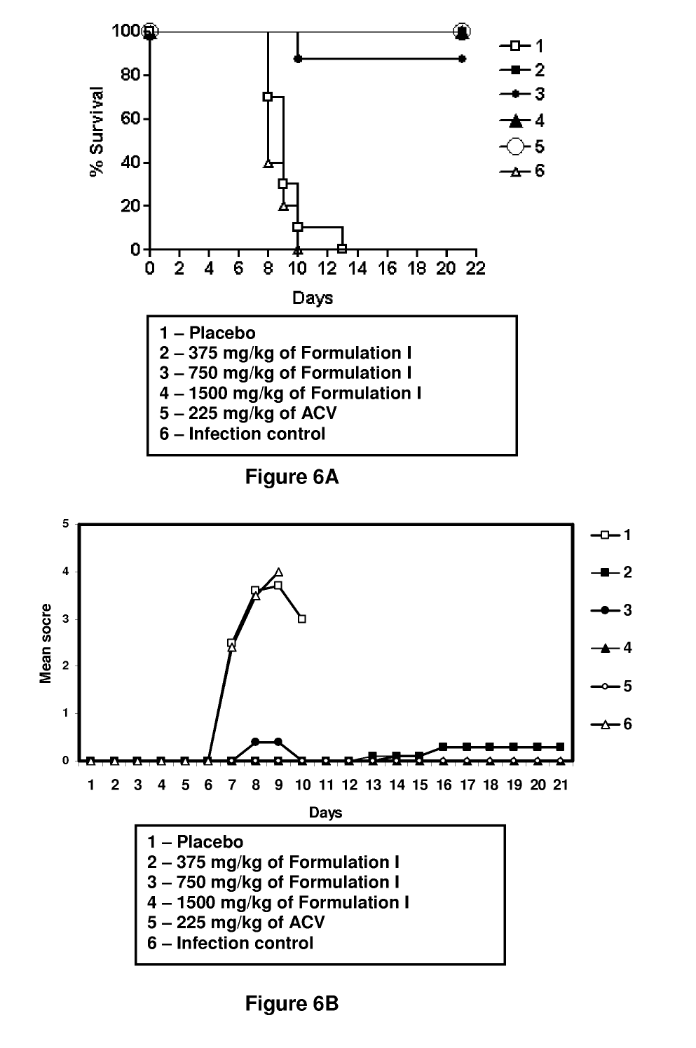

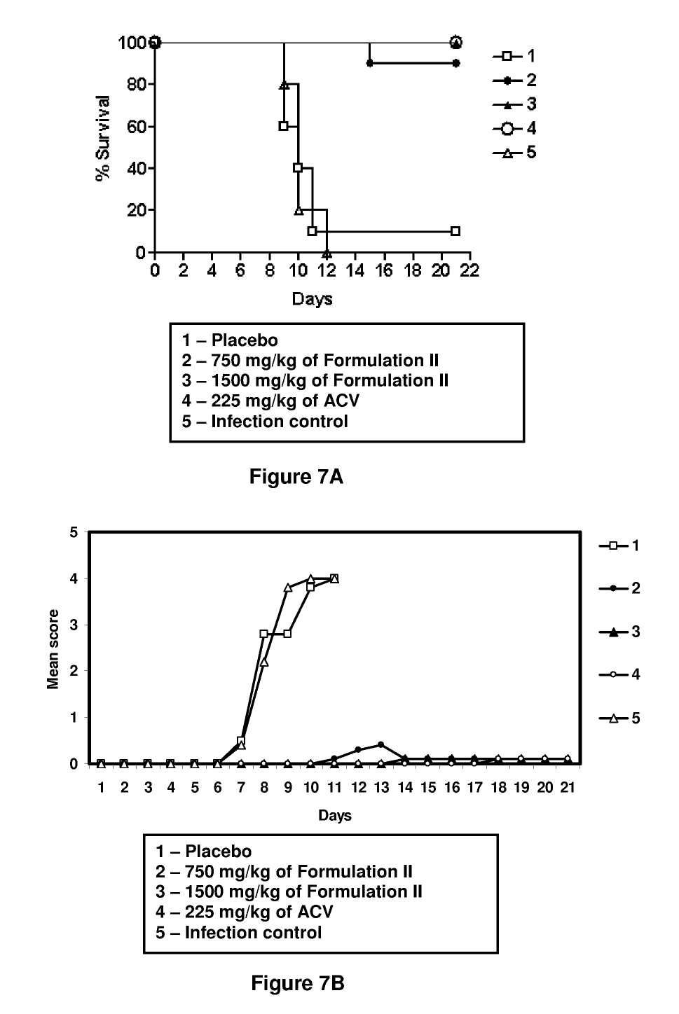

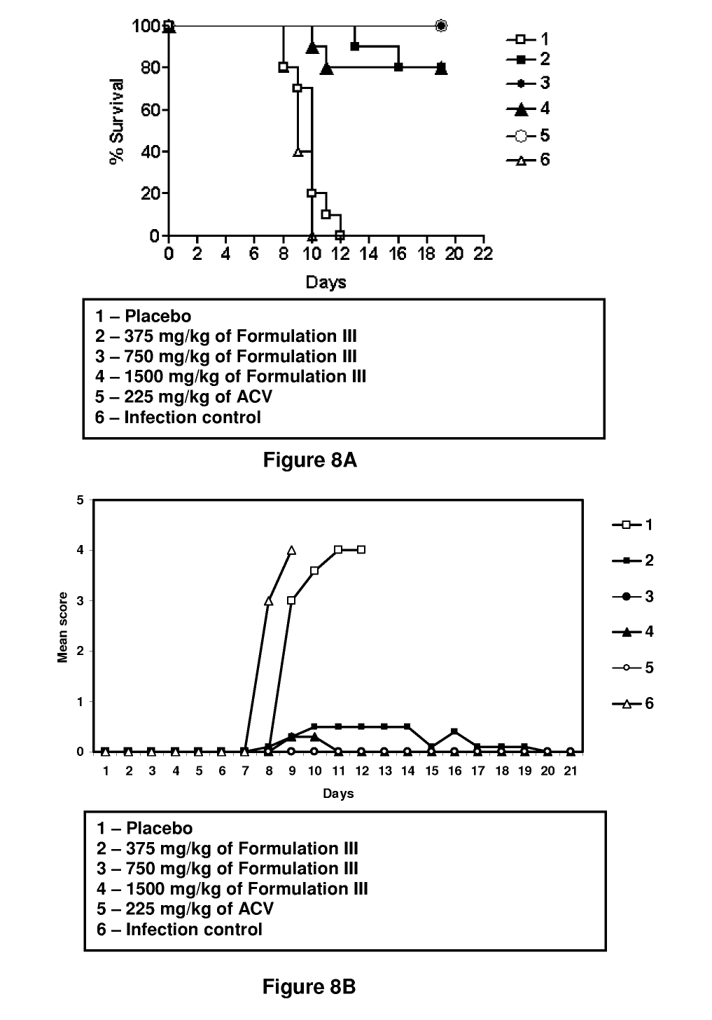

The observations noted were as follows:
1. Evaluation of extract of Example 1:
   a. Placebo treated group: The earliest sign of extravaginal infection appeared on day 5 and total mortality was observed in the animals.
   b. Acyclovir treated group: None of the mice showed extravaginal disease. The animals showed 100% survival rate.
   c. Extract of Example 1 treated group:
      (i) Animals treated with 375 mg/kg dose of the extract: The earliest sign of extravaginal infection appeared on day 5 and all the survived animals clinically recovered by day 21. The survival rate was 30% in these animals.
      (ii) Animals treated with 750 mg/kg dose of the extract: The earliest sign of extravaginal infection appeared on day 5 and continued until the conclusion of the study at day 21. The survival rate was 50% in these animals.
      (iii) Animals treated with 1500 mg/kg dose of the extract: The earliest sign of extravaginal infection appeared on day 5 and all the survived animals clinically recovered by day 21. The survival rate was 40% in these animals.
      The results are depicted in FIG. 5A and FIG. 5B.
2. Evaluation of Formulation I:
   a. Placebo treated group: The earliest sign of extravaginal infection appeared on day 7 and total mortality was observed in the animals.
   b. Infection control group: The earliest sign of extravaginal infection appeared on day 7 and total mortality was observed in the animals.
   c. Acyclovir treated group: None of the mice showed extravaginal disease. The animals showed 100% survival rate.
   d. Formulation I treated group:
      (i) Animals treated with 375 mg/kg dose of the extract: The earliest sign of extravaginal infection appeared on day 13 and continued until the conclusion of the study at day 21. The survival rate was 100% in these animals.
      (ii) Animals treated with 750 mg/kg dose of the extract: The earliest sign of extravaginal infection appeared on day 8 and continued until the conclusion of the study at day 21. The survival rate was 87.5% in these animals.
      (iii) Animals treated with 1500 mg/kg dose of the extract: None of the mice showed extravaginal disease. The survival rate was 100% in these animals.
      The results are depicted in FIG. 6A and FIG. 6B.
3. Evaluation of Formulation II:
   a. Placebo treated group: The earliest sign of extravaginal infection appeared on day 9 and total mortality was observed in the animals.
   b. Infection control group: The earliest sign of extravaginal infection appeared on day 9 and total mortality was observed in the animals.
   c. Acyclovir treated group: The extravaginal disease appeared on day 18 and continued till day 21. The animals showed 100% survival rate.
   d. Formulation II treated group:
      (i) Animals treated with 750 mg/kg dose of the extract: The earliest sign of extravaginal infection appeared on day 11 and continued till day 21. The animals showed 90% survival rate.
      (ii) Animals treated with 1500 mg/kg dose of the extract: The earliest sign of extravaginal infection appeared on day 14 and continued till day 21. The animals showed 100% survival rate.
      The results are depicted in FIG. 7A and FIG. 7B
4. Evaluation of Formulation III:
   e. Placebo treated group: The earliest sign of extravaginal infection appeared on day 8 and total mortality was observed in the animals.
   f. Infection control group: The earliest sign of extravaginal infection appeared on day 8 and total mortality was observed in the animals.
   g. Acyclovir treated group: None of the mice showed extravaginal disease. The animals showed 100% survival rate.
   h. Formulation Ill treated group:
      (i) Animals treated with 375 mg/kg dose of the extract: The earliest sign of extravaginal infection appeared on day 8 and and continued until the conclusion of the study at day 21. The survival rate was 100% in these animals.
      (ii) Animals treated with 750 mg/kg dose of the extract: The animals did not exhibit any signs of extravaginal disease throughout the study. The survival rate was 100% in these animals.
      (iii) Animals treated with 1500 mg/kg dose of the extract: The animals did not exhibit any signs of extravaginal disease throughout the study. The survival rate was 80% in these animals.
      The results are depicted in FIG. 8A and FIG. 8B.

1-21. (canceled)

22. An isolated extract from roots of the plant Indigofera heterantha prepared by stirring the roots of the plant in a solvent in a ratio of 1:5 to 1:40 weight/volume for 2 hours to 24 hours at 40° C. to 50° C.; concentrating the extract; and optionally enriching the extract by solvent partitioning or chromatography.

23. A herbal composition comprising a therapeutically effective amount of an isolated extract of roots of the plant Indigofera heterantha, prepared according to claim 22, either alone or in combination with a pharmaceutically acceptable carrier.

24. A process for the preparation of the herbal composition as claimed in claim 23, comprising:
(a) preparing an extract from the roots of the plant Indigofera heterantha by stirring in a solvent in a ratio of 1:5 to 1:40 weight/volume for 2 hours to 24 hours at 40° C. to 50° C.;
(b) concentrating the solvent extract obtained in step (a);
(c) optionally drying the extract obtained in step (b) under high vacuum (0.01-5 mm Hg);
(d) optionally enriching the extract obtained in step (b) or step (c) using materials selected from polyamide resin, gelatin/sodium chloride solution, polyvinylpyrrolidone, caffeine, lead (II) acetate or hide powder;
(e) optionally enriching the extract obtained in step (b), step (c) or step (d) by solvent partitioning or chromatography; and
(f) optionally mixing the extract of step (b), step (c), step (d) or step(c) with a pharmaceutically acceptable carrier.

25. The process as claimed in claim 24, wherein the solvent for extracting the plant Indigofera heterantha is selected from methanol, ethanol, n-propanol, isopropanol, n-butanol, acetone, ethyl acetate, dichloromethane, water, and a mixture thereof.

26. The process as claimed in claim 25, wherein the solvent for extracting the plant Indigofera heterantha is a mixture of methanol and water, or ethanol and water.

27. The process as claimed in claim 24, wherein the material used for enriching the extract is polyamide resin.

28. The process as claimed in claim 24, wherein the solvents for enriching the extract by solvent partitioning arc selected from water, petroleum ether, dichloromethane, chloroform, ethyl acetate, methanol, acetone, acetonitrile, n-propanol, iso-propanol, and butanol or a mixture thereof.

29. The herbal composition as claimed in claim 23, wherein the herbal composition is formulated for oral or topical administration.

30. The herbal composition as claimed in claim 29, wherein the herbal composition is formulated for topical administration in the form of a cream, gel or ointment.

31. The herbal composition as claimed in claim 30, wherein the herbal composition comprises 5% to 50% (w/w) of the extract.

32. A method of treating viral infection comprising administering to a subject in need thereof, a herbal composition as claimed in claim 23.

33. The method as claimed in claim 32, wherein the viral infection is caused by HSV.

34. The method as claimed in claim 33, wherein the viral infection is caused by HSV-2.
